(19) United States
(12) Patent Application Publication
(10) Pub. No.: US 2026/0241134 A1
(43) Pub. Date: Aug. 20, 2026

(54) RAPID DECOMPRESSION AND HIGH-FLOW OXYGEN SUPPLY LARYNGOSCOPE

(71) Applicant: HADA MEDTECH INC, Markham (CA)

(72) Inventor: Da HA, Hohhot, Inner Mongolia (CN)

(21) Appl. No.: 19/161,000

(22) PCT Filed: Mar. 11, 2024
(86) PCT No.: PCT/CN2024/081038

§ 371 Date: Aug. 29, 2025

(30) Foreign Application Priority Data
Mar. 13, 2023 (CN) 202310248165.X
Mar. 13, 2023 (CN) 202320496450.9

(51) Int. Cl. A61M 16/20; A61B 1/00; A61B 1/267; A61M 39/00; A61M 39/10

(52) U.S. Cl. CPC A61M 16/202; A61B 1/00042; A61B 1/00068; A61B 1/00119; A61B 1/00128; A61B 1/00131; A61B 1/267; A61M 39/10; A61M 2039/0009; A61M 2039/1027; A61M 2202/0007; A61M 2202/0208; A61M 2205/3334; A61M 2205/3344; A61M 2210/1028

(57) ABSTRACT

Disclosed in the present invention is a rapid pressure-reducing and high-flow oxygen supply laryngoscope, comprising a laryngoscope handle, an extension arm, and a disposable high-transparency laryngoscope blade. An oxygen supply tube is provided in the laryngoscope handle and the extension arm; an oxygen source tube in communication with the oxygen supply tube is provided on the outer wall of the laryngoscope handle; and an oxygen flow limiting device is provided on the oxygen supply tube or the oxygen source tube. The present invention has the following advantages: the structure is simple, the manufacturing costs are low, and standardized industrial batch production is facilitated; during use, the laryngoscope is easy to operate, is convenient and rapid to use, and can be rapidly connected to central oxygen supply for use; the oxygen flow is limited, so that the high pressure oxygen supplied by the central oxygen supply is reduced from 400-700 kPa to 50-60 kPa, the oxygen flow is equal to or greater than 45 liters per minute, and high-flow oxygen supply can enable the airflow to generate an airway positive pressure effect, thereby increasing the end-expiratory lung volume of a patient, achieving lung recruitment maneuver, improving the lung compliance, enabling oxygen to be rapidly dispersed in the lung to improve the hypoxic state of the patient, and reducing the risk of hypoxia asphyxia due to intubation.

### TECHNICAL FIELD

The present invention relates to a rapid pressure-reducing and high-flow oxygen supply laryngoscope and belongs to the field of medical devices.

### BACKGROUND ART

The laryngoscope is a tool for exposing a voice box during tracheal intubation. For a patient with respiratory failure and an oxygen saturation below 30%, the safe time limit for tracheal intubation may not exceed 10 seconds, and the rescue success rate would be greatly reduced if it exceeds 10 seconds; for a patient with an oxygen saturation below 90% and requiring emergency intubation according to clinical diagnosis, the safe time limit for tracheal intubation may not exceed 40 seconds, the patient is at risk if it exceeds 40 seconds, and brain death of the patient would be caused if it exceeds 180 seconds. During intubation rescue, situations such as obesity, short neck, high larynx, protruding teeth, maxillo-facial injury, and the like are frequently encountered, making it difficult for healthcare workers to rapidly complete intubation and posing the greatest challenges and tests to anesthesia and emergency physicians. It has significant importance for improving the success rate of rescue and lowering morbidity and disability by adding a rapid oxygen supply device to the laryngoscope so as to enable rapid delivery of oxygen into the body during intubation, thereby changing oxygen-deprived intubation to oxygenated intubation, extending the safe time limit of intubation, reducing the risk of hypoxic asphyxia due to intubation, preventing brain hypoxia caused by intubation delays, securing a critical window for rescue and improving the rescue success rate.

The oxygen supply pressure into the airway may not exceed 50-60 kPa for adults and may not exceed 20-30 kPa for children. However, the central oxygen supply pressure in hospitals is in a range of 400-700 kPa. Directly connecting a laryngoscope to the central oxygen supply of a hospital can cause severe airway pressure injuries. Therefore, oxygen-supplying laryngoscopes are prohibited from being connected with the central oxygen supply of a hospital at 400-700 kPa.

If an oxygen-supplying laryngoscope is connected to the central oxygen supply of a hospital, it must be first connected to a pressure-reducing valve for adjusting the oxygen supply pressure through pressure adjustment, which reduces the central oxygen pressure from 400-700 kPa to 50-60 kPa. This operation is cumbersome and time-consuming, making it unsuitable for the critical and urgent nature of emergency intubation in critically ill patients.

Even when an oxygen-supplying laryngoscope is connected to a medical float-type flowmeter, the maximum oxygen flow provided by the float-type pressure-reducing flowmeter does not exceed 15-20 l/min, while the clinically defined high-flow oxygen supply is equal to or greater than 45 l/min for adults. The float-type flowmeter cannot meet the clinical requirements for high-flow oxygen supply.

### SUMMARY OF THE INVENTION

The object of the present invention is to provide a rapid pressure-reducing and high-flow oxygen supply laryngoscope that is directly connected to hospital central oxygen supply while ensuring the clinical requirements for high-flow oxygen supply, which can overcome the above-mentioned deficiencies.

The present invention is implemented by means of the following technical solution: a rapid pressure-reducing and high-flow oxygen supply laryngoscope, comprising a laryngoscope handle, an extension arm, and a disposable high-transparency laryngoscope blade, wherein an oxygen supply tube is provided in the laryngoscope handle and the extension arm; an oxygen source tube in communication with the oxygen supply tube is provided on the outer wall of the laryngoscope handle; and an oxygen flow limiting device is provided on the oxygen supply tube or the oxygen source tube.

Preferably, the oxygen flow limiting device is a flow limiting orifice plate disposed inside the oxygen supply tube or the oxygen source tube.

Preferably, the oxygen flow limiting device is a vent plug disposed inside the oxygen supply tube or the oxygen source tube.

Preferably, the oxygen flow limiting device comprises a three-way pipe structure, and an adjustment bolt is connected to sealing threads inside the middle pipe of the three-way pipe structure.

Preferably, a quick plug connector in communication with the oxygen supply tube is provided on the outer wall of the laryngoscope handle, and the oxygen source tube is correspondingly modularly plugged into the quick plug connector.

Preferably, a quick plug connector in communication with the oxygen supply tube is provided inside the laryngoscope handle, a movable sleeve is provided on the outer wall of the laryngoscope handle, and the opening of the movable sleeve inside the laryngoscope handle is correspondingly in contact with a release ring of the quick plug connector; a stop protrusion is provided on the sidewall of the opening of the movable sleeve inside the laryngoscope handle, a limiting ring is provided on the sidewall of the opening of the movable sleeve on the outside of the laryngoscope handle, and the oxygen source tube passes through the inner cavity of the movable sleeve and is correspondingly modularly plugged into the quick plug connector.

Preferably, an oxygen source control switch is provided on the oxygen supply tube.

Preferably, the oxygen source control switch is a manual push switch, and the switch button of the manual push switch is disposed on the outer wall of the laryngoscope handle.

Preferably, the oxygen source control switch is an electric control valve, and the switch button for controlling the opening and closing of the electric control valve is disposed on the outer wall of the laryngoscope handle.

The present invention has the following advantages: compared with the prior art, the structure is simple, the manufacturing cost is low, and standardized industrial batch production is facilitated; during use, the laryngoscope is easy to operate, is convenient and rapid to use, and can be rapidly connected to central oxygen supply for use; the oxygen flow is limited, so that the high pressure oxygen supplied by the central oxygen supply is reduced from 400-700 kPa to 50-60 kPa, the oxygen flow is equal to or greater than 45 liters per minute, and high-flow oxygen supply can enable the airflow to generate an airway positive pressure effect, thereby increasing the end-expiratory lung volume of a patient, achieving lung recruitment maneuver, improving lung compliance, enabling oxygen to be rapidly dispersed in the lung to improve the hypoxic state of the patient, and reducing the risk of hypoxic asphyxia due to intubation.

### BRIEF DESCRIPTION OF THE DRAWINGS

FIG. 1 is a schematic diagram of the overall structure of Embodiment 1.

FIG. 2 is a schematic diagram of the overall structure of the laryngoscope handle in Embodiment 1.

FIG. 3 is a schematic diagram of the overall structure of a disposable high-transparency laryngoscope blade.

FIG. 4 is a schematic diagram of the oxygen flow limiting device being a flow limiting orifice plate in Embodiment 1.

FIG. 5 is an A-A cross-sectional view of FIG. 4.

FIG. 6 is a schematic diagram of the oxygen flow limiting device being a vent plug in Embodiment 1.

FIG. 7 is a B-B cross-sectional view of FIG. 6.

FIG. 8 is a schematic diagram of the oxygen flow limiting device being a three-way pipe structure in Embodiment 1.

FIG. 9 is a schematic diagram of the overall structure of Embodiment 2.

FIG. 10 is a schematic diagram of the overall structure of Embodiment 3.

FIG. 11 is a schematic diagram of installation of a movable sleeve.

FIG. 12 is a schematic diagram of the oxygen source control switch being a manual push switch in Embodiment 4.

FIG. 13 is a schematic diagram of the oxygen source control switch being an electric control valve in Embodiment 4.

FIG. 14 is a schematic diagram of the overall structure of Embodiment 5.

FIG. 15 is a schematic diagram of the overall structure of the laryngoscope handle in Embodiment 5.

FIG. 16 is a schematic diagram of the oxygen flow limiting device being a flow limiting orifice plate in Embodiment 1.

FIG. 17 is a C-C cross-sectional view of FIG. 5.

FIG. 18 is a schematic diagram of the oxygen flow limiting device being a vent plug in Embodiment 1.

FIG. 19 is a D-D cross-sectional view of FIG. 5.

FIG. 20 is a schematic diagram of the oxygen flow limiting device being a three-way pipe structure in Embodiment 5.

FIG. 21 is a schematic diagram of the overall structure of Embodiment 6.

FIG. 22 is a schematic diagram of the overall structure of Embodiment 7.

FIG. 23 is a schematic diagram of the oxygen source control switch being a manual push switch in Embodiment 8.

FIG. 24 is a schematic diagram of the oxygen source control switch being an electric control valve in Embodiment 8.

### DETAILED DESCRIPTION OF EMBODIMENTS

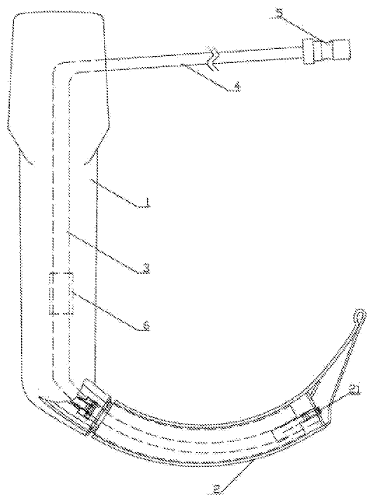

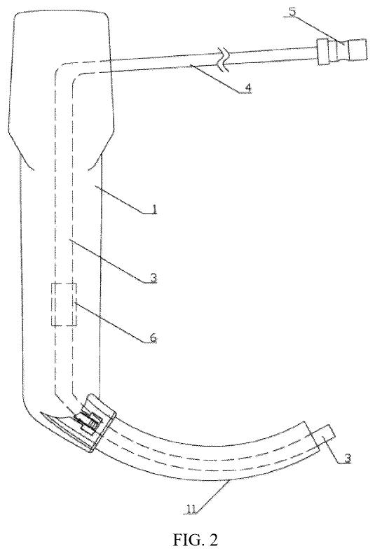

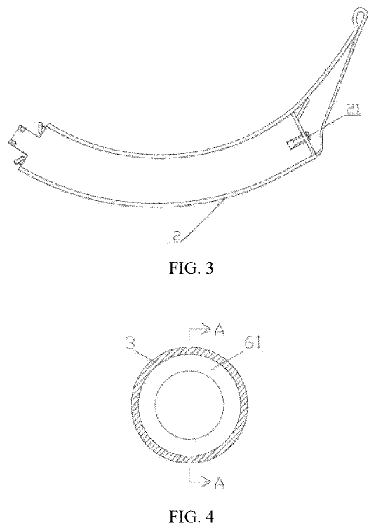

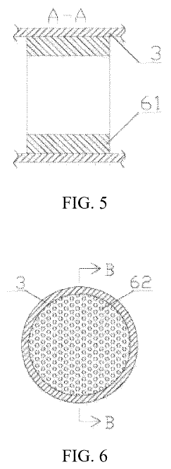

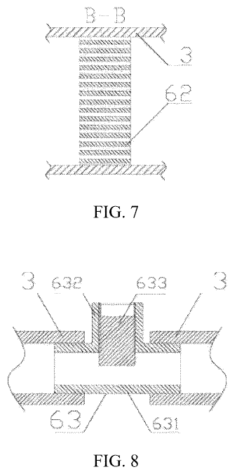

Embodiment 1: as shown in FIG. 1 to FIG. 3, a rapid pressure-reducing and high-flow oxygen supply laryngoscope comprises a laryngoscope handle 1 and a disposable high-transparency laryngoscope blade 2, the disposable high-transparency laryngoscope blade 2 is correspondingly modularly plugged on an extension arm 11 at one end of the laryngoscope handle 1, an oxygen supply tube 3 is provided in the laryngoscope handle 1 and the extension arm 11, the opening at one end of the oxygen supply tube 3 in the extension arm 11 passes through the outer wall of the top end of the extension arm 11 and is disposed at the outside of the top end of the extension arm 11, and an oxygen outlet 21 corresponding to the opening of the oxygen supply tube 3 at the top end of the extension arm 11 is provided on the disposable high-transparency laryngoscope blade 2; an oxygen source tube 4 in communication with the oxygen supply tube 3 is provided on the outer wall of the laryngoscope handle 1; a quick plug 5 for corresponding plug connection with a quick-connect interface of central oxygen supply is provided at the end of the oxygen source tube 4; an oxygen flow limiting device 6 is provided on the oxygen supply tube 3, and as shown in FIG. 4 and FIG. 5, the oxygen flow limiting device 6 is a flow limiting orifice plate 61 disposed inside the oxygen supply tube 3; or as shown in FIG. 6 and FIG. 7, the oxygen flow limiting device 6 is a vent plug 62 disposed inside the oxygen supply tube 3; or as shown in FIG. 8, the oxygen flow limiting device 6 is a three-way pipe structure 63, and two straight pipes 631 of the three-way pipe structure 63 are in communication with the oxygen supply tube 3; and an adjustment bolt 633 is connected to sealing threads inside the middle pipe 632 of the three-way pipe structure 63.

Instructions for use: in use, insert the quick plug 5 into a quick-connect interface of hospital central oxygen supply, the high-pressure oxygen at 400-700 kPa of the hospital central oxygen supply is reduced to a pressure of 50-60 kPa through the oxygen flow limiting device 6 on the oxygen supply tube 3 and ejected from the oxygen outlet 21 on the disposable high-transparency laryngoscope blade 2, thereby enabling the laryngoscope to deliver oxygen into the body during the intubation process to expose the voice box and reducing hypoxic asphyxia during intubation.

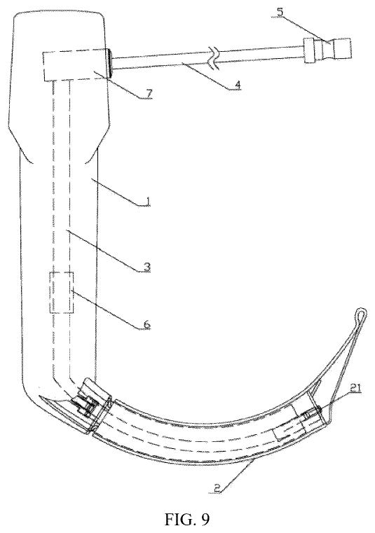

Embodiment 2: as shown in FIG. 9, a rapid pressure-reducing and high-flow oxygen supply laryngoscope has an overall structure that is the same as in Embodiment 1 with the difference thereof in that a quick plug connector 7 in communication with the oxygen supply tube 3 is provided on the outer wall of the laryngoscope handle 1, an oxygen source tube 4 is correspondingly modularly plugged into the quick plug connector 7, the oxygen source tube 4 is modularly plugged into the laryngoscope handle 1 via the quick plug connector 7 so as to facilitate the carrying and accommodation of the laryngoscope, and the quick plug connector 7 has simple operations and convenient quick connection and is easy for locking and unlocking the oxygen source tube 4.

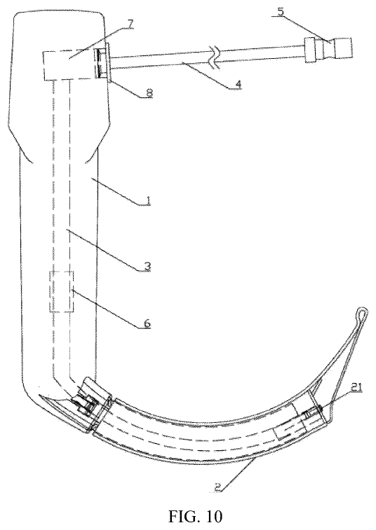

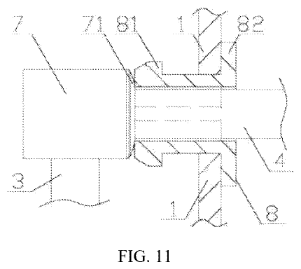

Embodiment 3: as shown in FIG. 10, a rapid pressure-reducing and high-flow oxygen supply laryngoscope comprises a laryngoscope handle 1 and a disposable high-transparency laryngoscope blade 2, it has an overall structure that is the same as in Embodiment 1 with the difference thereof in that a quick plug connector 7 in communication with the oxygen supply tube 3 is provided inside the laryngoscope handle 1, a movable sleeve 8 is provided on the outer wall of the laryngoscope handle 1, and as shown in FIG. 11, the opening of the movable sleeve 8 inside the laryngoscope handle 1 is correspondingly in contact with a release ring 71 of the quick plug connector 7; a stop protrusion 81 is provided on the sidewall of the opening of the movable sleeve 8 inside the laryngoscope handle 1, a limiting ring 82 is provided on the sidewall of the opening of the movable sleeve 8 on the outside of the laryngoscope handle 1, an oxygen source tube 4 passes through the inner cavity of the movable sleeve 8 and is correspondingly modularly plugged into the quick plug connector 7, the oxygen source tube 4 is modularly plugged onto the laryngoscope handle 1 via the quick plug connector 7 so as to facilitate the carrying and accommodation of the laryngoscope, and the quick plug connector 7 has simple operations and convenient quick connection and is easy for locking and unlocking the oxygen source tube 4; the movable sleeve 8 and the quick plug connector 7 are in cooperative connection, the movable sleeve 8 is pressed to unlock the oxygen source tube 4, and the operation is simple, convenient, and rapid.

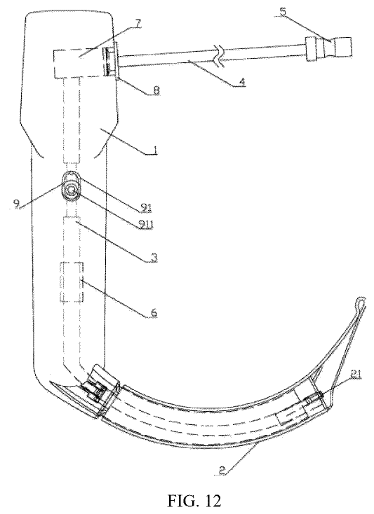

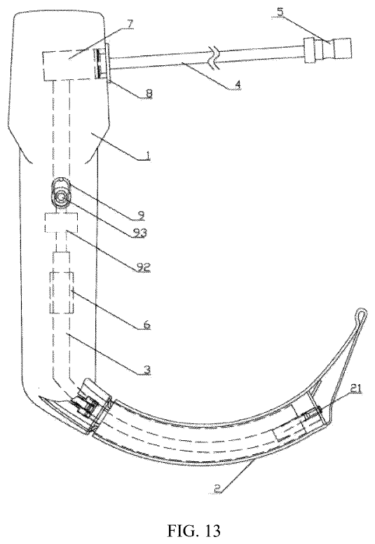

Embodiment 4: as shown in FIG. 12, a rapid pressure-reducing and high-flow oxygen supply laryngoscope comprises a laryngoscope handle 1 and a disposable high-transparency laryngoscope blade 2, it has an overall structure that is the same as in Embodiment 3 with the difference thereof in that an oxygen source control switch 9 is provided on the oxygen supply tube 3, the oxygen source control switch 9 is a manual push switch 91, and the switch button 911 of the manual push switch 91 is disposed on the outer wall of the laryngoscope handle 1; or as shown in FIG. 13, the oxygen source control switch 9 is an electric control valve 92, the switch button 93 for controlling the opening and closing of the electric control valve 92 is disposed on the outer wall of the laryngoscope handle 1, and with the oxygen source control switch 9, healthcare workers may switch the oxygen supply mode between a continuous oxygen supply mode and a jet oxygen supply mode by means of the oxygen source control switch 9.

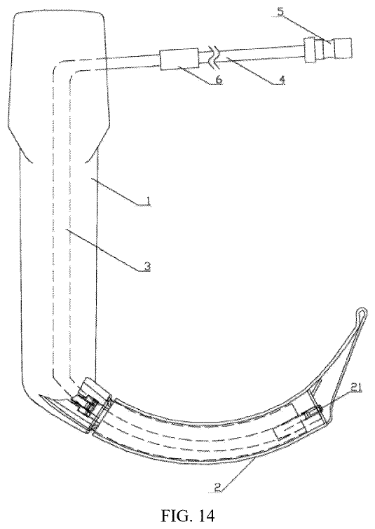

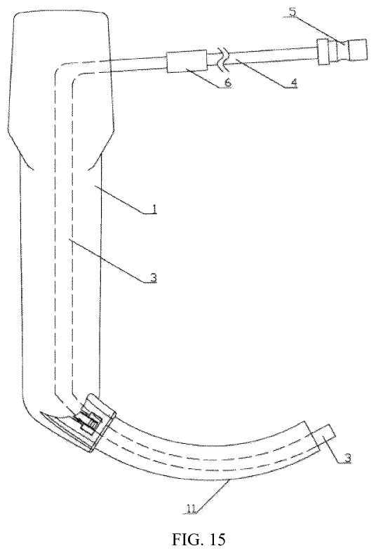

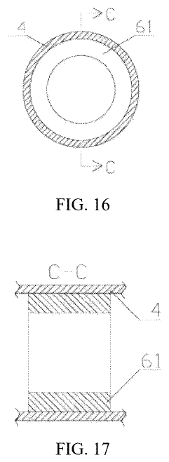

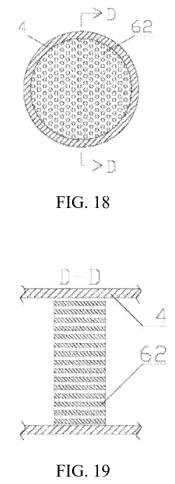

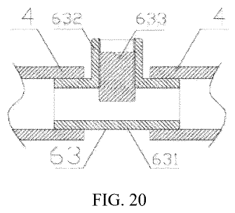

Embodiment 5: as shown in FIG. 14 and FIG. 15, a rapid pressure-reducing and high-flow oxygen supply laryngoscope comprises a laryngoscope handle 1 and a disposable high-transparency laryngoscope blade 2, the disposable high-transparency laryngoscope blade 2 is correspondingly modularly plugged on an extension arm 11 at one end of the laryngoscope handle 1, an oxygen supply tube 3 is provided in the laryngoscope handle 1 and the extension arm 11, the opening at one end of the oxygen supply tube 3 in the extension arm 11 passes through the outer wall of the top end of the extension arm 11 and is disposed at the outside of the top end of the extension arm 11, and an oxygen outlet corresponding to the opening of the oxygen supply tube 3 at the top end of the extension arm 11 is provided on the disposable high-transparency laryngoscope blade 2; an oxygen source tube 4 in communication with the oxygen supply tube 3 is provided on the outer wall of the laryngoscope handle 1; a quick plug 5 for corresponding plug connection with a quick-connect interface of central oxygen supply is provided at the end of the oxygen source tube 4; an oxygen flow limiting device 6 is provided on the oxygen source tube 4, and as shown in FIG. 16 and FIG. 17, the oxygen flow limiting device 6 is a flow limiting orifice plate 61 disposed inside the oxygen source tube 4; or as shown in FIG. 18 and FIG. 19, the oxygen flow limiting device 6 is a vent plug 62 disposed inside the oxygen source tube 4; or as shown in FIG. 20, the oxygen flow limiting device 6 comprises a three-way pipe structure 63, and two straight pipes 631 of the three-way pipe structure 63 are in communication with the oxygen source tube 4; and an adjustment bolt 633 is connected to sealing threads inside the middle pipe 632 of the three-way pipe structure 63.

Instructions for use: in use, insert the quick plug 5 into a quick-connect interface of hospital central oxygen supply, the high-pressure oxygen at 400-700 kPa of the hospital central oxygen supply is reduced to a pressure of 50-60 kPa through the oxygen flow limiting device 6 on the oxygen source tube 4 and ejected from the oxygen outlet 21 on the disposable high-transparency laryngoscope blade 2, thereby enabling the laryngoscope to deliver oxygen into the body during the intubation process to expose the voice box and reducing hypoxic asphyxia during intubation.

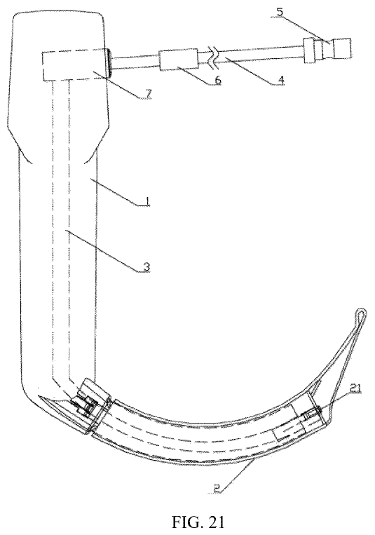

Embodiment 6: as shown in FIG. 21, a rapid pressure-reducing and high-flow oxygen supply laryngoscope comprises a laryngoscope handle 1 and a disposable high-transparency laryngoscope blade 2, it has an overall structure that is the same as in Embodiment 5 with the difference thereof in that a quick plug connector 7 in communication with the oxygen supply tube 3 is provided on the outer wall of the laryngoscope handle 1, and oxygen source tube 4 is correspondingly modularly plugged into the quick plug connector 7, the oxygen source tube 4 is modularly plugged into the laryngoscope handle 1 via the quick plug connector 7 so as to facilitate the carrying and accommodation of the laryngoscope, and the quick plug connector 7 has simple operations and convenient quick connection and is easy for locking and unlocking the oxygen source tube 4.

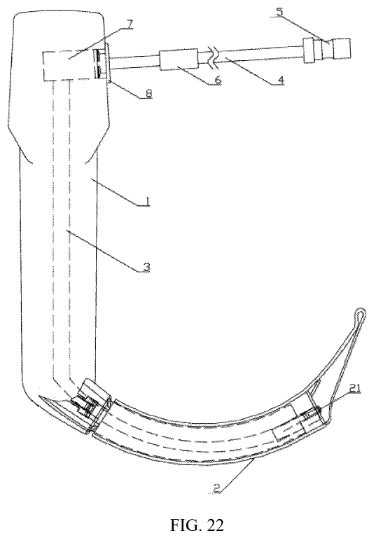

Embodiment 7: as shown in FIG. 22, a rapid pressure-reducing and high-flow oxygen supply laryngoscope comprises a laryngoscope handle 1 and a disposable high-transparency laryngoscope blade 2, it has an overall structure that is the same as in Embodiment 5 with the difference thereof in that a quick plug connector 7 in communication with the oxygen supply tube 3 is provided inside the laryngoscope handle 1, a movable sleeve 8 is provided on the outer wall of the laryngoscope handle 1, and as shown in FIG. 11, the opening of the movable sleeve 8 inside the laryngoscope handle 1 is correspondingly in contact with a release ring 71 of the quick plug connector 7; a stop protrusion 81 is provided on the sidewall of the opening of the movable sleeve 8 inside the laryngoscope handle 1, a limiting ring 82 is provided on the sidewall of the opening of the movable sleeve 8 on the outside of the laryngoscope handle 1, an oxygen source tube 4 passes through the inner cavity of the movable sleeve 8 and is correspondingly modularly plugged into the quick plug connector 7, the oxygen source tube 4 is modularly plugged onto the laryngoscope handle 1 via the quick plug connector 7 so as to facilitate the carrying and accommodation of the laryngoscope, and the quick plug connector 7 has simple operations and convenient quick connection and is easy for locking and unlocking the oxygen source tube 4; the movable sleeve 8 and the quick plug connector 7 are in cooperative connection, the movable sleeve 8 is pressed to unlock the oxygen source tube 4, and the operation is simple, convenient, and rapid.

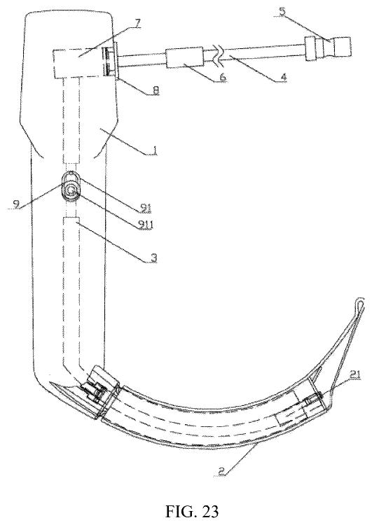

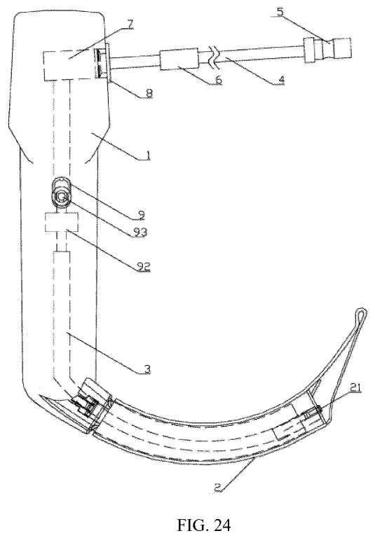

Embodiment 8: as shown in FIG. 23, a rapid pressure-reducing and high-flow oxygen supply laryngoscope comprises a laryngoscope handle 1 and a disposable high-transparency laryngoscope blade 2, it has an overall structure that is the same as in Embodiment 7 with the difference thereof in that an oxygen source control switch 9 is provided on the oxygen supply tube 3, the oxygen source control switch 9 is a manual push switch 91, and the switch button 911 of the manual push switch 91 is disposed on the outer wall of the laryngoscope handle 1; or as shown in FIG. 24, the oxygen source control switch 9 is an electric control valve 92, the switch button 93 for controlling the opening and closing of the electric control valve 92 is disposed on the outer wall of the laryngoscope handle 1, and with the oxygen source control switch 9, healthcare workers may switch the oxygen supply mode between a continuous oxygen supply mode and a jet oxygen supply mode by means of the oxygen source control switch 9.

1. A rapid pressure-reducing and high-flow oxygen supply laryngoscope, comprising a laryngoscope handle, an extension arm, and a disposable high-transparency laryngoscope blade, which is characterized in that an oxygen supply tube is provided in the laryngoscope handle and the extension arm; an oxygen source tube in communication with the oxygen supply tube is provided on the outer wall of the laryngoscope handle; and an oxygen flow limiting device is provided on the oxygen supply tube or the oxygen source tube, wherein the oxygen flow limiting device comprises a flow limiting orifice plate disposed inside the oxygen supply tube or the oxygen source tube to provide an oxygen flow output of at least 45 l/min and pressure of 50-60 kPa.

2. The rapid pressure-reducing and high-flow oxygen supply laryngoscope according to claim 1, wherein the flow limiting orifice plate is disposed inside the oxygen supply tube or the oxygen source tube.

3. The rapid pressure-reducing and high-flow oxygen supply laryngoscope according to claim 1, wherein the oxygen flow limiting device comprises a vent plug disposed inside the oxygen supply tube or the oxygen source tube.

4. The rapid pressure-reducing and high-flow oxygen supply laryngoscope according to claim 1, wherein the oxygen flow limiting device comprises a three-way pipe structure, and an adjustment bolt is connected to sealing threads inside the middle pipe of the three-way pipe structure.

5. The rapid pressure-reducing and high-flow oxygen supply laryngoscope according to claim 1, comprising a quick plug connector in communication with the oxygen supply tube provided on the outer wall of the laryngoscope handle, and the oxygen source tube is correspondingly modularly plugged into the quick plug connector.

6. The rapid pressure-reducing and high-flow oxygen supply laryngoscope according to claim 1, comprising:
a quick plug connector in communication with the oxygen supply tube provided inside the laryngoscope handle,
a movable sleeve provided on the outer wall of the laryngoscope handle, and the opening of the movable sleeve inside the laryngoscope handle is correspondingly in contact with a release ring of the quick plug connector;
a stop protrusion provided on the sidewall of the opening of the movable sleeve inside the laryngoscope handle, and
a limiting ring provided on the sidewall of the opening of the movable sleeve on the outside of the laryngoscope handle, and the oxygen source tube passes through the inner cavity of the movable sleeve and is correspondingly modularly plugged into the quick plug connector.

7. The rapid pressure-reducing and high-flow oxygen supply laryngoscope according to claim 5, comprising an oxygen source control switch provided on the oxygen supply tube.

8. (canceled)

9. The rapid pressure-reducing and high-flow oxygen supply laryngoscope according to claim 7, wherein the oxygen source control switch is a manual push switch, and comprising a switch button of the manual push switch disposed on the outer wall of the laryngoscope handle.

10. The rapid pressure-reducing and high-flow oxygen supply laryngoscope according to claim 7, wherein the oxygen source control switch is an electric control valve, and comprising a switch button for controlling the opening and closing of the electric control valve disposed on the outer wall of the laryngoscope handle.
